(19) United States
(12) Patent Application Publication
(10) Pub. No.: US 2006/0142729 A1
(43) Pub. Date: Jun. 29, 2006

(54) Adult incontinence garment with extension strip

(75) Inventors: Daniel M. Sivilich, Freehold, NJ (US); Paul S. Rankin, Bowling Green, OH (US)

Correspondence Address: EPSTEIN DRANGEL BAZERMAN & JAMES, LLP, 60 EAST 42ND STREET, SUITE 820, NEW YORK, NY, 10165, US

(73) Assignee: Humanicare International, Inc.

(21) Appl. No.: 11/070,742

(22) Filed: Mar. 2, 2005

Related U.S. Application Data
(63) Continuation-in-part of application No. 11/022,502, filed on Dec. 27, 2004, now abandoned.

(51) Int. Cl. A61F 13/15

(52) U.S. Cl. 604/395

(57) ABSTRACT

The an adult incontinence garment has front and rear body portions connected by a crotch portion and pressure sensitive adhesive tabs for connecting the front and rear body portions to one another when in place on an individual of normal girth. An extension strip is provided for adapting the garment for use by an individual of larger than normal girth. The extension strip is received between the body portions. It includes an adhesive layer on one side of and a plastic layer adapted to receive the adhesive tabs on the other side. The tab-receiving layer is formed of material that permits the tabs to be received in a readily detachable and re-attachable manner so that the garment can be readily monitored and adjusted while in place.

### CROSS-REFERENCE TO RELATED APPLICATIONS

This application is a continuation-in-part of copending application Ser. No. 11/022,502, filed Dec. 27, 2004.

### STATEMENT REGARDING FEDERALLY SPONSORED RESEARCH OR DEVELOPMENT

Not Applicable

### REFERENCE TO A “SEQUENCE LISTING”, A TABLE, OR A COMPUTER PROGRAM LISTING APPENDIX SUBMITTED ON COMPACT DISC

Not Applicable

### BACKGROUND OF THE INVENTION

1. Field of the Invention

This invention relates to absorbent garments such as diapers, particularly for use by adults as incontinence garments, in order to render such garments useable on persons of greater than normal girth and to render such garments more readily useable under the conditions to which they are normally subjected.

2. Description of Prior Art Including Information Disclosed Under 37 CFR 1.97 AND 1.98

Incontinence garments generally comprise first and second body portions connected by a crotch portion. The first and second body portions are adapted to be disposed at the front and back of the individual user. The body portions are secured in place by adhesive tabs extending from one of the body portions and adapted to be secured to the other of the body portions.

Incontinence garments are usually manufactured to standard adult population sizing. However, the standard adult size garment, or the largest size when a plurality of sizes is provided, may not necessarily be large enough in girth to fit unusually large individuals within their age groupings, such as obese individuals or bariatric patients. The purpose of the present invention is to enable a garment of standardized size, which is designed to fit an individual of normal girth, to be used by a variety of different size individuals of considerably greater than normal girth.

Further, adult incontinence garments are normally subjected to adverse conditions involving feces and/or urine. Even under ideal circumstances, they are also often a source of discomfort to the wearer. It is therefore often desirable, once such a garment has been secured in place on the wearer, to be able to release that securement for monitoring or adjustment purposes and then re-secure the garment in place. It is another object of the present invention to provide the ability to repeatedly detach and re-attach the securing tabs when required.

We are aware of a product designed to permit the adjustment of the girth of an infant diaper that is marketed by SCA North America, 500 Baldwin Tower, Eddystone, Pa. 19022. However, that product has limited applicability as it can only be used with specially constructed garments that employ a hook and loop securement means, requiring the tabs to be fabricated of either hook or loop material. Because the hook or loop materials are almost impossible to clean when soiled due to the irregular nature of the surfaces, it is often not considered ideal for use for adult incontinence garments.

### SUMMARY OF THE INVENTION

The adult incontinence garment of the type to which the present application pertains is secured in place on the patient by adhesive tabs that extend from the side edges of one of the body portions. Those tabs are designed to overlap the side edges of the other body portion and to be adhesively secured to that other body portion.

That adhesive securement normally involves the use of a layer of pressure sensitive adhesive on the securement tabs. However, when pressure sensitive adhesive is used as a means of securement, the relatively fragile nature of the garment material itself makes the release and re-attachment of the pressure sensitive adhesive tabs problematic. This is because the portion of the garment surface to which the tabs are attached tends to tear when the tabs are detached, making re-attachment in that area impossible.

That problem is overcome in the present invention, while permitting a conventional adult incontinence garment to fit a variety of different size individuals of greater than normal girth, by providing the garment with a pair of extension strips. Each of the extension strips is designed to be secured to the standard adhesive securing tabs that extend at either side of the garment between the body portions of the garment. This enables the garment portions to be secured to one another as in conventional, but be separated from each other to a greater than normal extent, so as to fit the larger individual.

In addition, the area of the extension strip designed to be engaged by the normal pressure-sensitive-adhesive-bearing securing tabs is provided with a surface of appreciable dimension to which the pressure sensitive adhesive on the normal securing tabs of the garment is to be attached. That surface has the characteristic that when a tab is once adhesively attached thereto, the tab may be readily detached and re-attached without damage to the extension strip. In that way, the ability to readjust and monitor the garment is provided.

### BRIEF DESCRIPTION OF THE DRAWINGS

To the accomplishment of the above, and to such other objects as may hereinafter appear, the present invention relates to an absorbent garment and an extension strip for use therewith as described in this specification, and as disclosed in the accompanying drawings, in which:

FIG. 1 is a front elevation view of the garment with the strip of the present intention in place on the patient;

FIG. 2 is a three-quarter prospective exploded view of such a garment and of the unattached extension strip to be used therewith;

FIG. 3 is a detail view of the normal securing tab-extension strip engagement;

FIG. 4 is a fragmentary detail view of a portion of the extension strip with the affixed exposed plastic layer partially in place;

FIG. 5 is a view similar to FIG. 1 but showing one possible attachment sequence with the extension strip ready to be engaged with the first body part of the garment; and

FIG. 6 illustrates the next step in the sequence of attachment to accomplish the finished condition of FIG. 1.

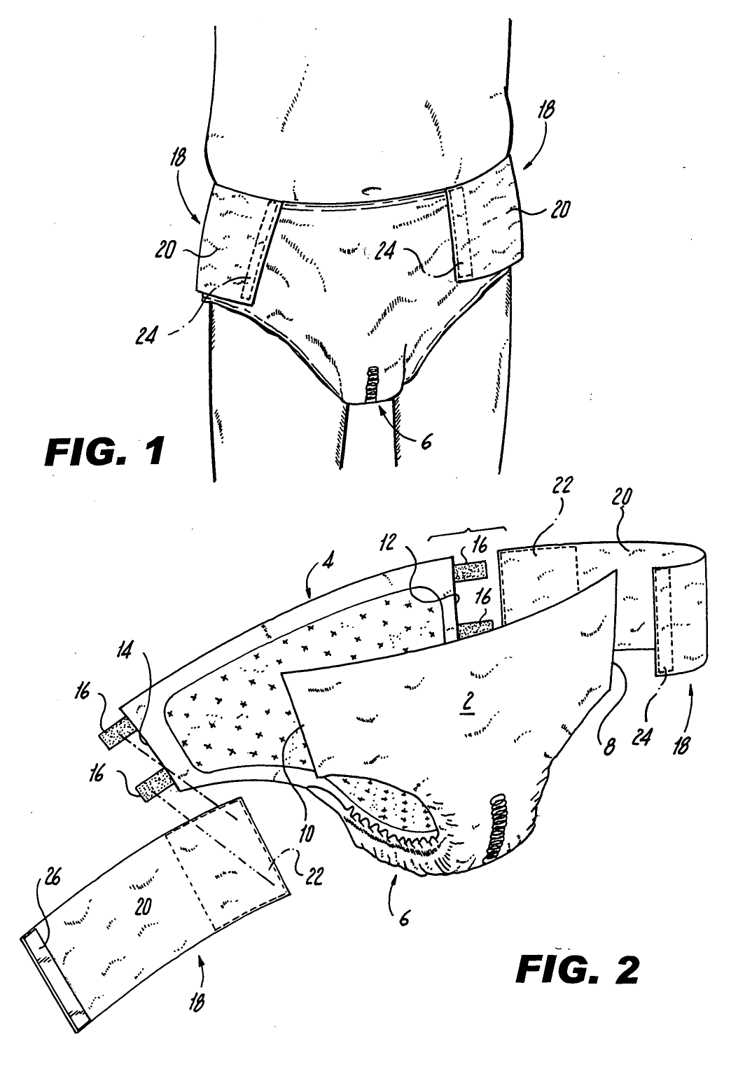

### DETAILED DESCRIPTION OF THE PREFERRED EMBODIMENTS

A typical incontinence garment for use by adults with excretion problems comprises a first or front absorbent body portion generally designated 2 adapted to extend over the patient's lower abdomen, a second or rear absorbent body portion generally designated 4 adapted to extend over the rear of the patient's lower body portion, and an absorbent crotch portion generally designated 6 connected between the lower ends of the body portions 2 and 4.

Body portion 2 has side edges 8 and 10. Body portion 4 has side edges 12 and 14. When the garment is in place, side edges 8 and 12 are on one side of the patient and side edges 10 and 14 are on the other side of the patient. The rear body portion 4 at its side edges 12 and 14 is provided with outwardly extending tabs 16. The lower surfaces of tabs 16 are provided with a layer of pressure sensitive adhesive. When the garment is situated on the wearer, tabs 16 are adapted to be pressed against the surface of the front body section 2 in the vicinity of its side edges 8 and 10, thereby to secure the garment in place.

Only a very limited degree of adjustability is possible with this system to enable the garment to fit properly on a given individual. This is because the tabs are relatively short in length and reliability of securement requires that a significant portion of the tabs 16 engage the exterior surface of the front body portion 2, if the garment is to reliably stay in position, as it must.

The body sections 2 and 4 and the crotch section 6 each will typically comprise a liquid-impermeable exterior surface sheet, a body-facing liquid-permeable interior sheet and an enclosed absorbent body. The structure and compositional details of those elements may be varied widely, as is understood in the trade.

In one particularly pertinent example the exterior surface sheet, to which the tabs 16 are normally adhesively attached, is made of relatively flimsy plastic film. Because of that, once the garment has been secured in place on the wearer, the tabs 16 cannot be removed from the front body portion 2 without causing damage to the exterior surface sheet. Since it is usually desired that the exterior surface sheet be liquid-impermeable, damage to that sheet can be a serious matter.

In accordance with the present invention, a conventional adult incontinence garment, designed for use by an individual of normal size, and therefore incapable of being used by an individual whose girth exceeds the norm, can be used by such an individual by utilizing the extension strip of the present invention, generally designated 18. That strip comprises a length of flexible material such as woven or non-woven fabric, flexible film, wood pulp products (paper, tissue, etc.), foils and the like, all tensile materials. The term “tensile materials” as here used characterizes material that is capable of withstanding the stresses to which it may be subjected in use without substantial permanent distortion or damage. It includes, without limitation, material that may stretch elastically but continues to assert a restoring force when stretched.

Strip 18 is designed to be attached to a conventional adult incontinence garment of the type having pressure sensitive adhesive tabs permanently affixed to and extending from one of the body portions to secure that body portion to the other body portion. The strips 18 as here specifically disclosed in their preferred form comprise a length 20 of appropriate tensile material having at one end and on one surface a preferably thin layer 22 which may be formed of an appropriate plastic, foil, coated paper or the like, and having at its other end, and preferably on its other surface, a layer 24 of pressure sensitive adhesive. That adhesive layer 24 is preferably covered, before the strip 18 is used, with a strip 26 of release material made of paper, silicone treated paper, plastic film, or the like.

The layer 22 is of a material to which the securing tabs 16 of the garment will adhesively stick with sufficient force so that the garment, when once applied, will reliably stay in place, but from which the tabs 16 may be removed without tearing or damage and then reapplied. The length of the layer 22 in the direction of the length of the strip 18 is preferably appreciable, thus providing leeway for adapting the garment to the size of the individual.

This is particularly important for bed-ridden patients being cared for by another. The care-giver can open the garment to check if it is soiled and then close it back up when that has been done, without damaging the liquid impermeable exterior surface of the garment, and in addition the size of the garment can readily be expanded or contracted by the care-giver to accommodate the patient's need, desire or condition.

The adhesive layer 24 at an end of the strip 18 may be of the same type as is provided on the garment tabs 16. It is designed to be secured to the exterior surface sheet of the front panel 2 of the garment.

In a preferred embodiment, 30 gm/m² 12″ wide spun bond polypropylene is used as a base web for the strip 18. A ¾″ wide pressure sensitive adhesive transfer tape (Ludlow Tape #7328) is applied to one side of the web to produce the adhesive strip 24. A 4″ wide bi-axially oriented polypropylene film, coated on one side with a pressure sensitive adhesive (Avery Dennison BOPP Frontal Tape) is applied to the underside of the opposite edge of the web to produce the layer 22. Other plastics, such as polyethylene and polyester, are readily available for use in place of polypropylene. The web is then cut into 7″ lengths along the web machine direction.

Typically, the preferred method of use of the present invention may is as follows: The conventional adult incontinence garment proper is first positioned on the body of the wearer. With individuals of larger than normal girth, there will be a gap between the edges 8 and 12, and between the edges 10 and 14, such that the tabs 16 extending from the rear body portion 4 either will not reach the exterior surface of the front body portion 2 or will not overlap the exterior surface of front body 2 sufficiently to provide a secure attachment.

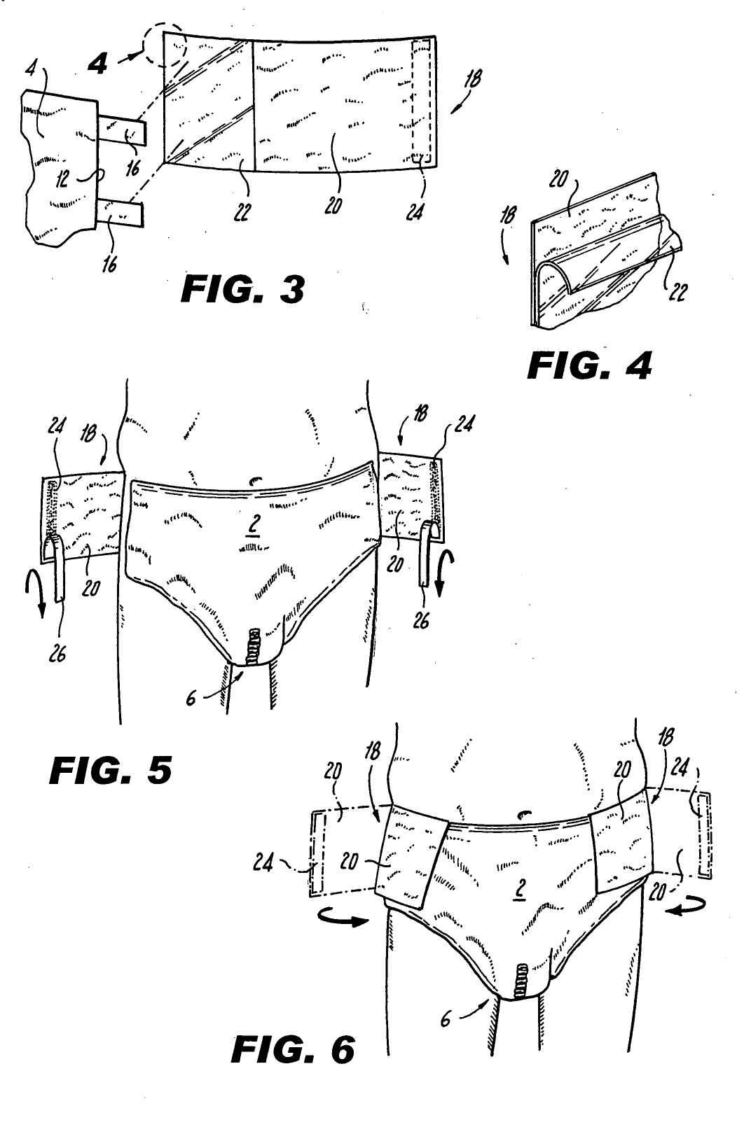

In that case, extension strips 18 are put into place and secured to extend between the side edges of the body portions 2 and 4. As here specifically disclosed, and as illustrated in particular in FIGS. 3 and 6, the strips 18 may be first secured to the rear body portion 4 by causing the garment's normal securing tabs 16 to press against and become adhesively secured to layer 22 of the strip. The other end of the strip 18 may then be moved forward (FIG. 5), the release strips 26 may be removed to expose the adhesive layers 24, and as indicated in FIG. 6, the free ends of the strips 18 may be moved over the front body section 2 and pressed down against it so as to cause the adhesive layers 24 to engage the exterior surface sheet of the front body portion 2 As has been indicated, if re-attaching or monitoring is required, the tabs 16 can be removed from layer 22 of the extension strips, the garment can be manipulated for monitoring or adjustment, and the tabs 16 can be reattached to the layers 22. Of course it is entirely possible to reverse the sequence of use, to first secure the adhesive slayer 24 to the front body portion 2 and then secure the normal tabs 16 to the layers 22.

If in extreme cases the strips 18 are not adequate to accommodate a particularly obese patient, additional strips 18 can be used in tandem, by attaching one strip to another to further extend the effective size of the garment. It is also possible to use only a single extension strip 18 at only one side of the garment, but the imbalance thus provided would probably result in some discomfort on the part of the user.

When, as described above, the strip 20 is one foot long and when two such strips are used the effective girth of the garment will be increased by approximately 21 inches. Also, the strip ends could engage the front body panel 2 could do so on the inner surface of the body section, with adhesive layer 24 on the outer surface of the strip, but it is thought that there would be less possibility of skin irritation with the strip 18 on the exterior surface of the body portion 2 as illustrated.

It will now be appreciated that the present invention relates to extension strips that enable a conventional adult incontinence garment with adhesive connecting tabs to be used on a variety of different size wearers of larger than normal girth. This is accomplished by fabricating the extension strips to include a plastic layer adapted to receive the adhesive tabs in a releasable and re-attachable fashion on one side and a pressure sensitive adhesive layer on the other side.

While only a limited number of preferred embodiments of the present invention have been here disclosed for purposes of illustration, it will be apparent that many variations and modifications may be made therein. It is intended to cover all of those variations and modifications that fall within the scope of the present invention, as defined in the following claims:

We claim:

1. An adult incontinence garment comprising first and second body portions connected by a crotch portion, said body portions having side edges, and at least one pressure adhesive bearing tab fixed to and extending from one of said body portions normally operative to secure said body portion to said other body portion, with the garment in place on a user of normal girth, and in combination therewith, an extension strip normally separate from and adapted when in use to extend between said first and second body portions, said extension strip comprising a layer adapted to receive said pressure sensitive adhesive tab and a layer of pressure sensitive adhesive to secure said strip to said first and second body portions respectively, said strip when thus secured extending between the side edges of said body portions, thereby enabling said garment to fit an individual of greater than normal girth.

2. The garment of claim 1, in which said tab-receiving layer of said extension strip comprises a material capable, when once secured, of release from and re-securement to said tab.

3. The garment of claim 1, in which said extension strip has first and second ends and in which said extension strip adhesive layer is proximate one end of said strip and said tab-receiving layer is proximate the other end of said strip.

4. The garment of claim 3, in which said strip has two opposite surfaces, said tab-receiving layer being located on one of said surfaces and said adhesive layer being located on the other surface.

5. The garment of claim 1, in which said tab-receiving layer of said extension strip comprises a material selected from the group consisting of polypropylene, polyethylene and polyester.

6. The garment of claim 1 further comprising means for permanently securing said tab-receiving layer to said extension strip.

7. In the garment of claim 1 where said extension strip comprises first and second surfaces and wherein said tab-receiving layer and said adhesive layer are situated on opposite ones of said surfaces.

8. The garment of claim 1 wherein said tab-receiving layer and said adhesive layer of said extension strip are spaced from each other along said extension strip.

9. The garment of claim 1 wherein said tab-receiving layer is of appreciable dimension in the direction of length of said extension strip, thereby to enable substantial girth adjustment of said garment.

10. The garment of claim 5 wherein said tab-receiving layer is of appreciable dimension in the direction of length of said extension strip, thereby to enable substantial girth adjustment of said garment.

11. The absorbent garment of claim 10 wherein said extension strip has two opposite surfaces and wherein said tab-receiving layer is located on one of said surfaces and said adhesive layer is located on the other of said surfaces.

12. The absorbent garment of claim 11 wherein said tab-receiving layer and said adhesive layer are spaced from each other along said extension strip.

13. An extension strip for adapting an adult incontinence garment to fit an individual of larger than normal girth comprising a length of tensile material having first and second ends, carrying at said first end pressure sensitive adhesive material and carrying at its second end an exposed layer.

14. The extension strip of claim 13 wherein said exposed layer is of appreciable dimension in the direction of the length of said strip.

15. The extension strip of claim 13 in which said strip has first and second opposite surfaces with said pressure sensitive adhesive on said first surface and said exposed layer on said second surface.

16. The extension strip of claim 14 wherein said strip has first and second opposite surfaces with said pressure sensitive adhesive on said first surface and said layer on said second surface.

17. The extension strip of claim 13 wherein said exposed layer comprises material selected from the group consisting of polypropylene, polyethylene and polyester.

18. The extension strip of claim 14 wherein said exposed layer comprises material selected from the group consisting of polypropylene, polyethylene and polyester.

19. The extension strip of claim 15 wherein said exposed layer comprises material selected from the group consisting of polypropylene, polyethylene and polyester.

20. The extension strip of claim 16 wherein said exposed layer is composed of material selected from the group consisting of polypropylene, polyethylene and polyester.

21. The extension strip of claim 13 further comprising means for permanently fixing said exposed layer to said extension strip.
